(19) United States
(12) Patent Application Publication
(10) Pub. No.: US 2023/0248028 A1
(43) Pub. Date: Aug. 10, 2023

(54) APPARATUS AND METHOD FOR TREATING FOODSTUFF CONTAINERS

(71) Applicant: Red Bull GmbH, Fuschl am See (AT)

(72) Inventors: Klemens HANS, Eugendorf (AT); Daniel HERZOG, Giebhubl (AT)

(73) Assignee: Red Bull GmbH, Fuschl am See (AT)

(21) Appl. No.: 18/011,986

(22) PCT Filed: Jun. 18, 2021
(86) PCT No.: PCT/EP2021/066698

§ 371 Date: Dec. 21, 2022

(30) Foreign Application Priority Data
Jun. 26, 2020 (AT) A 60204/2020
Oct. 30, 2020 (EP) 20000392.9

(51) Int. Cl. A23L 3/04; A61L 2/26; A61L 2/18; B65B 55/02; A23L 3/00; B08B 9/20

(52) U.S. Cl. CPC A23L 3/04; A61L 2/26; A61L 2/18; B65B 55/02; A23L 3/001; A23L 3/003; B08B 9/20; A61L 2101/06

(57) ABSTRACT

The invention relates to an apparatus for treating foodstuff containers by spraying with a biocide-containing treatment liquid in at least one treatment zone (Z1-Z4) equipped with spraying means (B) for spraying the containers, the apparatus further comprising transport means for transporting the containers through the treatment zones (Z1-Z4), at least one generator (G1) for producing a biocide solution, at least one heating device for heating the treatment liquid, lines for supplying the biocide solution from the generator (G1) to the treatment zones (Z1-Z4) and, optionally, to at least one further biocide consumer device (BV1, BV2), lines for supplying the treatment liquid to the treatment means (B), as well as valves (V1-V7), controllable metering devices and pumps required for controlling the liquid flows, characterized in that
a) the apparatus comprises at least two generators (G1, G2) for producing the biocide solution, and
b) the generators (G1, G2) are connected or connectable neither directly nor indirectly to one another, but via corresponding feed lines to all treatment zones (Z1-Z4) and to any further biocide consumer device (BV1, BV2); and a method for operating such an apparatus.

The present invention relates to an apparatus and a method for treating tightly sealed foodstuff containers with a treatment liquid.

### RELATED ART

Conserving foodstuffs within tightly sealed containers, e.g., fruit and vegetable juices or cans, soft drinks or energy drinks, is usually achieved by pasteurization. Therefore, containers will be heated to 60° C. or up to 100° C. (high-temperature pasteurization) for a short time, in order to kill vegetative phases of microorganisms. Compared to sterilization, which is done at temperatures above 100° C., this is a very mild method by which the consistency and ingredients of the foodstuff and therefore also their taste will hardly be affected.

Pasteurization as such will usually be performed by spraying a correspondingly pre-heated aqueous treatment liquid in pasteurizing apparatuses with different configurations, e.g., a tunnel-type pasteurizer, plate-type pasteurizer or chamber pasteurizer. Due to continuously recycling and re-using the aqueous treatment liquid, it will become more and more contaminated over time. In addition to the elevated temperatures and a high relative humidity of the ambient air, this will promote the proliferation of microorganisms within the treatment liquid itself and may cause its contamination, which may lead to the formation of biofilms on the foodstuff containers which are to be pasteurized. To prevent such contaminations and the formation of biofilms, disinfectants or biocides such as chlorine dioxide are added to the treatment liquid.

Due to their limited stability, such biocide solutions used within the treatment liquid are produced in situ in special biocide generators of the pasteurization apparatus; in case of chlorine dioxide this will be done, for example, by using the chlorine-chlorite process, the hydrochloric acid-chlorite process or the peroxodisulfate-chlorite process. Such apparatuses and corresponding treatment methods are disclosed in WO 2016/100996 A1, WO 2016/100997 A1, DE 10 2018 205 009 A1 and the related WO 2019/192817 A2 as well as in a yet unpublished European Patent Application by the applicant. Of these applications, WO 2016/100996 A1 as well as WO 2016/100997 A1 both describe the option of envisioning a plurality of generators—therein referred to as “chemical sources”—for the production of biocide solutions, which generators, however, are all “line-connected” to the same metering device, so that the added dosing of any biocide solutions produced within the generators will still be done via the single metering device and will thus occur at the same point of the apparatus.

It is a disadvantage of all of these embodiments that in case of malfunctions or during maintenance of the one or more biocide generators or of the metering device connected thereto, the whole pasteurizing apparatus must be temporarily decommissioned until respective works have been completed.

Thus, it is the objective of the invention to improve the known pasteurizing and similar apparatuses for treating foodstuff containers in order to avoid such deactivations.

### DISCLOSURE OF THE INVENTION

This objective will be achieved by the present invention in a first aspect by providing an apparatus for treating tightly sealed foodstuff containers by spraying with a treatment liquid containing at least one biocide in at least one treatment zone equipped with a plurality of spraying means for spraying the containers, the apparatus further comprising means of transportation for transporting the containers through the treatment zones, at least one generator for producing a solution of at least one biocide, at least one heating device for heating the treatment liquid, feeding lines for delivering the biocide solution from the generator to the at least one treatment zone and optionally to at least one more biocide consumer device as well as any valves required for controlling liquid flows, adjustable metering devices and pumps, the inventive apparatus being characterized in that:
a) the apparatus comprises at least two generators for producing the biocide solution, and
b) the generators are connected or can be connected neither directly nor indirectly with one another, but via corresponding feed lines with all treatment zones and any further biocide consumer devices.

By providing at least a second generator for producing the biocide solution, it is ensured that, in the event of failure of a single generator, the operation of the entire apparatus, which is usually operated continuously and with a high throughput, must not be completely stopped.

However, the present invention does not simply provide an apparatus comprising at least two generators all of which feed the flow of biocide solution generated therein into a common storage tank and/or into a single metering device—each of which cases would represent an “indirect connection” between the generators—, from which all treatment zones and other biocide consumer devices are subsequently fed, as indicated in WO 2016/100996 A1 and WO 2016/100997 A1. This would not solve the problem mentioned at the beginning, since this single storage tank or the single metering device could also fail and also has to be maintained regularly, which in turn would result in a temporary shutdown of the apparatus.

Instead, according to the present invention, all, i.e., at least two, generators of the apparatus, being neither directly nor indirectly connected to one another, are connected or can be connected via corresponding feed lines to all treatment zones and other biocide consumer devices. As a result, their supply with the biocide solution can be continued even in the event of a failure or maintenance of a generator; this completely eliminates the need to shut down the apparatus and ensures its reliability in this respect.

In addition, in this way, all treatment zones and other biocide consumer devices can be supplied with biocide solution from only one biocide generator or from several biocide generators at the same time, which offers various other advantages. For example, only a portion of the treatment zones can be supplied with biocide solution from all generators for a given period of time, while the remaining treatment zones can be shut down for cleaning or maintenance. Further advantages, including the ability to produce biocide solutions with different parameters in each of the individual generators, are discussed in more detail below.

The biocide solution produced in one generator is prevented from flowing into another by the sheer fact that the biocide solutions are ejected from each generator at sufficient pressure by means of an integrated pump (which is therefore not shown as such in the figures). In preferred embodiments of the invention, a check fitting such as a check valve or a check flap, preferably a check valve, is additionally provided at the outlet of each generator. In this way, in the event of a pump malfunction, a backflow of the biocide solution produced by the respective generator into the latter is prevented, but also an influx of biocide solution produced in another generator is completely excluded.

In this context, “biocide consumer device” means all devices, equipment and components of the apparatus according to the invention, to which biocide solution is to be supplied at least occasionally or continuously in order to control harmful organisms, in particular microbes and fungi. “Controlling” herein means disinfection in the narrower sense, i.e., a reduction in the number of bacteria by a factor of 10⁻⁵, as well as reductions to a lesser extent and the restriction of their proliferation, e.g., the prevention of the aforementioned germination or biofilm formation. Consequently, the treatment zones for the containers themselves are of course also “biocide consumers”, while further biocide consumer devices may be selected, for example, from devices for rinsing, washing, filling and sealing the containers, as well as for lubricating the transport means such as transport belts (“belt lubrication”) for the containers.

The feed lines leading from the generators to the biocide consumers are preferably connected or connectable to one another via mixing valves such as three-way, four-way or multi-way mixers, whereby the biocide solution from the individual generators may optionally be supplied to all or only some of the biocide consumers. In addition, fresh water may be supplied to the apparatus at the same time, preferably when four-way mixers are used, in order to compensate for water losses during operation.

In further preferred embodiments of the invention, the treatment liquid is an aqueous solution of the at least one biocide, and the generators are adapted to produce an aqueous solution of the at least one biocide with a variable concentration, higher than the biocide concentration of the treatment liquid, and are provided with respective feed lines for one or more biocide precursor solutions and optionally for fresh water and other additives to the biocide solution produced.

Even more preferably, the generators are used for the production of an aqueous chlorine dioxide solution, in particular by the peroxodisulfate chlorite process, and therefore each have feed lines for aqueous solutions of peroxodisulfate and of chlorite from corresponding storage tanks. However, chlorine dioxide produced by the chlorine-chlorite or hydrochloric acid-chlorite process and other suitable biocides, including disinfectants, are, of course, also encompassed by the invention.

In preferred embodiments, the apparatus according to the invention is used for pasteurization of the foodstuffs enclosed in the containers by means of contact with a hot treatment liquid and, for this purpose, comprises at least one heating device for heating the treatment liquid to above 60° C., preferably to at least 80° C. Particularly preferably, the treatment zones of the apparatus according to the invention are components of a conventional tunnel-type pasteurizer, plate-type pasteurizer or chamber pasteurizer, in particular a tunnel-type pasteurizer.

The apparatus according to the invention preferably further comprises measuring sensors for the temperatures and/or the concentrations of the liquid flows, which are particularly preferably connected in each case to a controller for controlling the heating devices, metering devices, pumps and/or valves on the basis of the measured values.

As mentioned above, biocide solutions with different parameters, e.g., with different temperatures and/or biocide concentrations, but also different quantities of biocide solution, can be produced in the individual devices in an apparatus according to the invention. In this way, different parameters of the biocide solution can be varied for some of the treatment zones and other biocide consumers, or even for all of them, without having to change the operating conditions of the generators, or at least only those of one generator.

In further particularly preferred embodiments, the apparatus further comprises one or more mixing chambers for mixing the biocide solution produced in at least one generator with fresh water in order to dilute it, and/or for mixing at least two biocide solutions produced by different generators before they enter the respective treatment zone or other biocide consumer devices. For example, when producing biocide solutions with different temperatures and/or biocide concentrations or different biocide solution volume flows in different generators, this allows for bringing the biocide solution to a desired value (temperature, concentration, quantity) before being supplied to the biocide consumers, without the need to produce a biocide solution with exactly this value in all generators. Moreover, by supplying fresh water to such a mixing chamber, the amount of liquid consumed by evaporation during the treatment of the containers can be replenished—especially in embodiments where the consumed treatment liquid is collected and recycled.

Mixing chambers may be provided, for example, instead of mixing valves described above, especially instead of four-way or multi-way mixers, to ensure more thorough mixing of multiple flows of biocide solution and/or of one or more biocide solution flows with fresh water. In any case, mixing chambers are not to be understood herein as collection or buffer vessels or reservoirs, which could also be considered as indirect connections between generators. Rather, the mixing chambers according to the present invention are operated dynamically, i.e., the sum of the incoming fluid flows per time unit is equal to that of the outgoing flows.

In further preferred embodiments of the apparatus according to the invention, it therefore further comprises:
one or more feed lines for fresh water, which are optionally connected to a fresh water reservoir; and/or
means for collecting and, if necessary, also for recycling used operating liquid; and/or
further heating or cooling means; and/or
means for exhausting the atmosphere surrounding the treatment zones.

In this way, the treatment apparatus can be adapted to various desired conditions in a wide variety of ways and thus optimized. For example, the biocide concentration of the treatment liquid can be subsequently reduced by dilution of one or more flows of biocide solution from one or more generators, e.g., in one or more mixing chambers or directly in feed lines to the treatment zones.

Collecting the used treatment liquid is a standard procedure when using the apparatus for the treatment of containers in conventional pasteurizers such as tunnel-type, plate-type or comb-type pasteurizers, and its recycling reduces the costs for operating the apparatus. Generally, the temperature of the containers is also controlled by repeated spraying with recycled treatment liquid, since, for example, a treatment liquid preheated to at least 80° C. on entering a treatment zone cools down further each time it comes into contact with a container. For this reason, the treatment media in some areas of a treatment zone are usually supplied exclusively with fresh, hot treatment liquid, while those in other areas are fed with treatment liquid collected below the containers, e.g., below a conveyor belt, and already cooled, which thus represents a recycling thereof. In this way, for example, a temperature curve or gradient of the containers within a treatment zone can be set without having to heat or cool the treatment liquid supplied to a zone to different temperatures.

By exhausting the atmosphere around the treatment zones, it can be purified before being released into the environment, which is especially necessary when using an aqueous chlorine dioxide solution as a biocide solution. And heating or cooling of biocide solution or of fresh water fed to supplement the amount of liquid allows (even more precise) control of the temperature in the treatment zones or in other biocide consumer devices.

In a second aspect, the present invention also provides a method of treating tightly sealed food containers by spraying with a treatment liquid containing at least one biocide using an apparatus according to the first aspect, the method comprising the steps of:
preparing a solution of the at least one biocide in at least two generators that are not directly or indirectly connected to one another;
directing the biocide solution to at least one treatment zone equipped with a plurality of treatment means, and optionally to at least one further biocide consumer device, each generator being connected or connectable to all treatment zones and any further biocide application devices;
mixing the treatment liquid with the biocide solution; and
spraying the containers with the treatment liquid via the spraying means as they are transported through the treatment zones.

In this way, of course, the same advantages over the state of the art can be achieved as described above for the apparatus according to the invention: the process can be operated continuously without the need to shut down the apparatus in the event of a generator failure, and, moreover, biocide solutions with different parameters can be produced in the individual generators.

Preferably, the containers are sprayed with an aqueous solution of at least one biocide, and an aqueous solution of at least one biocide with a variable concentration is produced in the generators, preferably an aqueous chlorine dioxide solution by the peroxodisulfate-chlorite process from peroxodisulfate and chlorite.

In preferred embodiments, the foodstuffs enclosed in the containers are pasteurized with the treatment liquid, which for this purpose is heated to above 60° C., preferably to at least 80° C., before the containers are sprayed. Furthermore, in preferred embodiments, the temperature and concentrations of the biocide solutions and the treatment liquid are measured, and the supply of the biocide solution and treatment liquid to the treatment zones and any further biocide consumer devices is controlled based on the measured values.

As was also already mentioned, aqueous biocide solutions with different concentrations can be produced in the generators at least periodically, which makes the handling of the containers in the individual treatment zones and the supply of further biocide consumers more flexible. It is particularly preferable to mix biocide solutions from different generators with different biocide concentrations and/or with different volumetric flows before they are fed into a treatment zone or before they are supplied to another biocide consumer, e.g., in one or more mixing chambers. In this way, the biocide solution can be brought to a predetermined concentration value, or a more precisely controllable amount of biocide solution can be supplied per time unit to some or all of the biocide consuming devices without requiring all of the devices to produce a biocide solution with exactly these values.

Furthermore, in preferred embodiments of the process according to the invention, by analogy with the apparatus according to the first aspect,
fresh water is supplied continuously or intermittently; and/or
used treatment liquid is collected and sometimes recycled; and/or
the treatment liquid is heated or cooled before it enters the spraying means; and/or
the atmosphere surrounding the treatment zones is exhausted,
   whereby again in each case the advantages described above with respect to the apparatus can be achieved.

Furthermore, in preferred embodiments of the method according to the invention, spraying liquid at different temperatures can be introduced into the individual spraying means of a treatment zone, with spraying liquid at the highest temperature being introduced particularly preferably into the spraying means located in the center of a treatment zone. In particular, the treatment liquid is heated to at least 80° C. before entering a treatment zone and is introduced into the spraying means located in the center of the zone, whereupon it cools down during the spraying of the food containers, is collected below the containers and is recycled by being introduced into the spraying means located at the start and at the end of the zone. In addition, the treatment liquid introduced into the spraying means at the start and end of the zone can be collected below the containers and recycled for renewed spraying of containers in these areas, the treatment liquid being mixed with the biocide solution produced in the generators only once it has cooled down to a predetermined temperature during spraying of the containers.

In this way, a temperature profile or gradient of the containers can be set without having to heat or cool the treatment liquid supplied to the treatment zone to different temperatures, and at the same time the use of biocide is limited. For example, the treatment liquid can be preheated to at least 80° C. throughout and fed to the spraying means located in the center of a treatment zone to heat the containers there to the highest temperature and to pasteurize the foodstuff contained therein. However, this temperature would be too high for a solution of chlorine dioxide, which is preferably used as a biocide, since chlorine dioxide already begins to decompose above 45° C. However, as the treatment liquid is recycled, it cools down further with each pass until it reaches a temperature suitable for contact with biocide. It must be taken into account that the biocide solution is considerably cooler than the treatment liquid, for example at room temperature, so that the treatment liquid is cooled further when mixed with the biocide solution.

This treatment liquid to which biocide was added can then be used for spraying the containers via spraying means at the beginning and end of the treatment zone, as seen in the transport direction. In this way, the containers, which are transported e.g., by a conveyor belt, are first preheated with the mixture of cooled treatment liquid and biocide solution, which has a temperature of 30-40° C., for example, after their entry into a treatment zone, and their surface is treated with biocide. Subsequently, their contents are pasteurized with hot treatment liquid, which may also consist exclusively of hot water, after which they are again sprayed with cooler treatment liquid containing biocide, the temperature of the treatment liquid recycled for this purpose being increased again upon contact with the hot containers and that of the containers being lowered at the same time. This means that the containers with pasteurized contents are already cooled to a desired extent before they leave the treatment zone.

### BRIEF DESCRIPTION OF THE DRAWINGS

The invention is described in more detail below with reference to non-limiting preferred examples and with reference to the accompanying drawings showing the following:

FIG. 1 is a schematic view of a relatively simple embodiment of an apparatus and method according to the present invention; and

FIGS. 2 and 3 are schematic views of further preferred embodiments of the invention with somewhat more complex interconnection.

### EXAMPLES

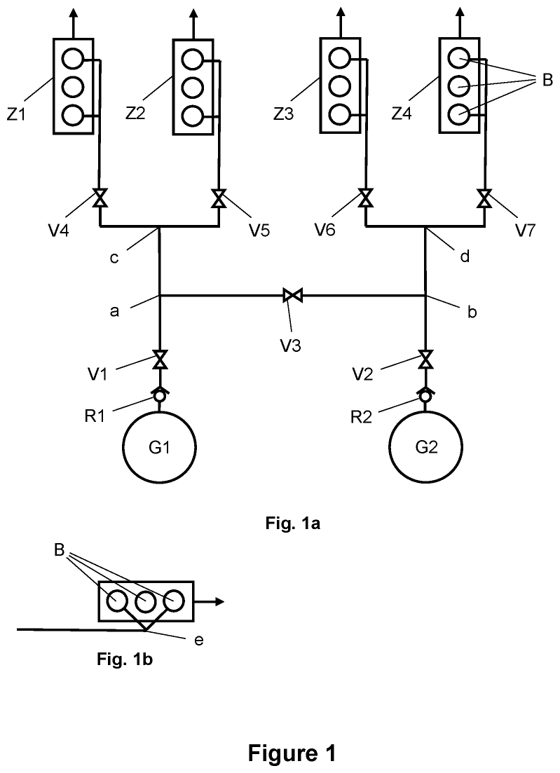

As mentioned above, FIG. 1 schematically shows a comparatively simple embodiment of the apparatus and the method of the present invention, wherein, however, only the lines and valves necessary for the connection of the components actually shown are drawn, but not, for example, the pumps also necessary for the operation of the apparatus, as well as heating and cooling devices, further valves, controllable metering devices, regulators and controllers. For a person skilled in the art, the components to be provided for smooth operation result quite naturally from the text description. With regard to the transport of liquids through the apparatus, it is assumed herein, for example, that a pump with the required pumping power is integrated in each component from which a liquid flow is delivered, such as in generators or storage tanks.

In particularly preferred embodiments, the present invention also relates to operating tunnel-type pasteurization systems in which a hot treatment liquid is used for pasteurizing foodstuffs, to which biocide solution from the generators is added. Therefore, it is further assumed herein that the apparatus comprises at least one heating device, not shown, in which water is heated to high temperatures, e.g., to 80-90° C., in order to feed the spraying means, in particular the spraying means in the center of the respective treatment zones, and to heat the food containers transported thereunder past the spraying means to above 60° C., preferably to at least 70° C., even more preferably to about 80° C. Sometimes, evaporators can also be used to heat the containers with steam to the desired temperature, which will condense on the containers and is in turn collected and recycled below the conveyor belt.

It should also be mentioned that, for simplicity and clarity, only embodiments with two generators are specifically illustrated and discussed herein. However, it is self-evident for the skilled person that the present invention also includes embodiments with more than two generators, where the corresponding components are to be connected analogously in each case.

FIG. 1a now shows a first simplified flow diagram based on two generators G1, G2, each of which is followed by a check fitting R1, R2 downstream. These are preferably check valves, e.g., spring-loaded ball check valves, but check flaps or backflow flaps can also be used to effectively prevent the biocide solution from flowing back into the generator and thus at the same time the influx of biocide solution produced in another generator, even if the integrated pump should not generate the required pressure due to a malfunction or the like.

The biocide solution produced in the generators, which is in particular an aqueous biocide solution, is pumped to a plurality of—herein four—treatment zones Z1 to Z4, each comprising a plurality of—herein three—treatment means B represented by circles O.

It is to be understood that neither the number of treatment zones nor that of the treatment means contained therein is limited thereto. In practice, as previously indicated, each of the treatment zones discussed and illustrated herein particularly preferably represents a tunnel-type pasteurizer, i.e., a tunnel usually being several meters long, in which the foodstuff containers, such as beverage bottles or cans, are placed while they remain therein or are placed, e.g., on a conveyor belt, are supplied from above with a hot liquid from a large number of supply means. According to the state of the art, several tunnel-type pasteurizers—for example, three each according to DE 10 2018 205 009 A1 cited above—are also supplied with treatment liquid by a biocide generator. The maximum number depends primarily on spatial and structural conditions and on the performance of the generator and the pumps, and will generally hardly be more than five. The number of sprayers per treatment zone or tunnel-type pasteurizer, which may be spray nozzles or atomizers, is of course also dependent on the above conditions.

The treatment zones Z1 to Z4 can each be preceded by a metering device (not shown) which controls the amount of biocide solution supplied to the respective treatment zone, e.g., a computer-controlled pump or the like, wherein this supply may be continuous or intermittent (“pulsed metering”).

In FIG. 1a, a valve V1 or V2 is provided in flow direction immediately after the check valves R1 and R2, by means of which the supply of biocide solution produced in the respective generator to the apparatus can be completely interrupted. Then, in the embodiment shown here, the inlet of the biocide solution from the respective other generator is shown, where the connection line also comprises a valve, V3, with which the connection can be interrupted, as needed.

Subsequently, the lines leading from each generator to the treatment zones branch out so that, when the valve V3 is closed, each of the generators G1 and G2 can supply only two treatment zones, i.e., Z1 and Z2 or Z3 and Z4, with treatment liquid. Each of the lines to the individual treatment zones Z1 to Z4 comprises a further valve, V4 to V7, by means of which the supply of treatment liquid to the respective treatment zone can be interrupted.

The spraying means B, to which the biocide solution is subsequently fed by means of a controllable metering device, if necessary, are preferably connected in parallel, as shown in FIG. 1a. This means that they are supplied with biocide solution at approximately the same pressure. It is also preferred that the supply to the spraying means B is as shown in FIG. 1b, which ensures the same pressure in all lines with the same supply volume. For example, a computer-controlled metering device can be provided at the position marked with “e”.

FIGS. 1a and 1b also show a preferred embodiment of the invention in which the biocide solution supplied from the generators is not fed into all spraying means B, but only into those at the beginning and end of the treatment zones, i.e., into the first and third spraying means B. In this case, it is assumed that the biocide solution supplied from the generators is not fed into all spraying means B, but only into those at the beginning and end of the treatment zones. It is assumed that the spraying agents B located in the middle of the treatment zones are sprayed (or sprayed) with treatment liquid of the highest temperature, e.g., with hot water at 80-90° C. Since mixing the biocide solution with such hot water would cause the biocide, especially chlorine dioxide, to decompose, mixing takes place only after the water used as the treatment liquid has been reduced by recycling, i.e., by collecting the chlorine dioxide that is present on the surface of the water to be treated. This means that the water that has run down the surface of the containers to be treated is collected below the conveyor belt and the containers at the beginning and end of the treatment zone are sprayed again.

Furthermore, multi-way valves can be provided at the branching positions a to d of the lines in addition to or preferably as an alternative to valves V1 to V7. For example, valve V3 can be replaced by two multi-way valves at positions a and b, and/or valves V4 to V7 can be replaced by multi-way valves at positions c and d. This provides the additional advantage that the valves V3 can be replaced by two multi-way valves at positions a and b, and/or valves V4 to V7 can be replaced by two multi-way valves at positions c and d. This offers the additional advantage that, when four-way mixers are used, fresh water can be introduced into the respective line at the respective position, for example, to compensate for liquid losses or to dilute the biocide solution, if necessary. However, the inlet of a line supplying fresh water or the like is not limited to multi-way valves at these positions, but can also take place at any other position within the plant.

Furthermore, for example, instead of some or all of the multi-way mixers, a dynamically operated mixing chamber, as described above, can be provided in each case to improve mixing, which can be advantageous especially if more than two flows need to be mixed.

In practice, e.g., in tunnel-type pasteurizers particularly preferred according to the invention, the treatment liquid formed by mixing the aqueous biocide solution with the sufficiently cooled water is recycled several times and finally drawn off from the treatment zones, as indicated by the arrows, and—optionally after appropriate purification or treatment—either removed from the treatment zones, or mixed with the water. If necessary, after appropriate cleaning or treatment, it is either disposed of, reused for spraying containers, or fed to other biocide consumers.

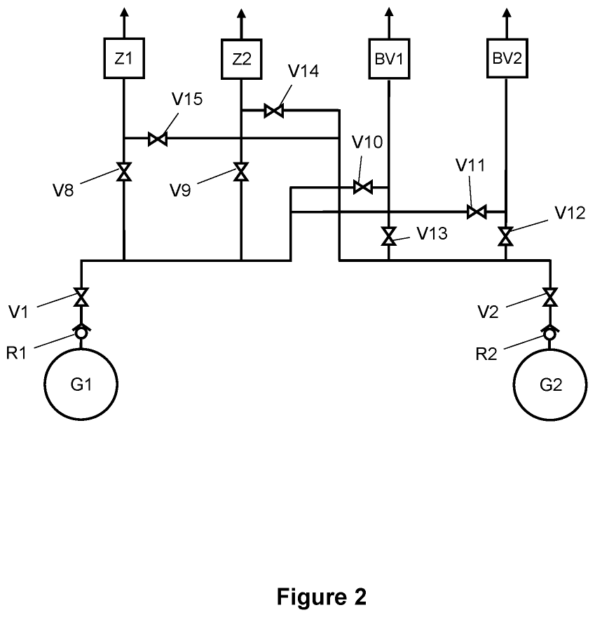

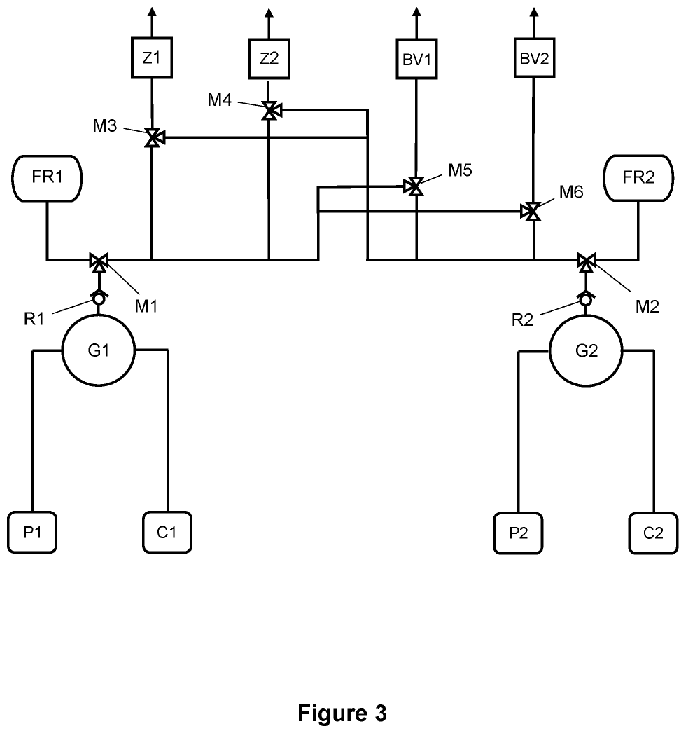

FIG. 2 shows another embodiment of the apparatus or process according to the invention as in FIG. 1, in which again two generators G1 and G2, but now only two treatment zones (e.g., tunnel-type pasteurizers) Z1 and Z2, as well as two further biocide consumer devices BV1 and BV2, supply biocide solution produced therein. For the sake of simplicity, we have omitted here—as well as in the following FIG. 3—the explicit representation of the individual spraying means within the treatment zones.

Additional biocide consumers in conventional systems for treating foodstuff containers are, above all, devices for rinsing, washing, filling and sealing the containers, as well as devices for lubricating the transport means, e.g., one or more transport belts for the containers (“belt lubrication”). These are also supplied—continuously or intermittently—with biocide solution produced in the generators, if necessary, after dilution of the same with fresh water or other treatment.

Another main difference to the embodiment of FIG. 1 is that the biocide solution supplied by the generators G1 and G2 to the treatment zones Z1 and Z2 and to the additional biocide consumers BV1 and BV2 is fed in this case directly upstream of the respective biocide consumers Z1, Z2, BV1 and BV2, so that in the case of simultaneous operation of both generators G1 and G2, the supply of biocide solution from one of them can also be interrupted for only one biocide consumer, whereas in the case of the alternative shown in FIG. 1 this is only possible in pairs. This means that it can be set individually for each biocide consumer whether it is to be supplied with biocide solution from one or from both generators—or, in the case of more than two generators, from several or from all generators. In this way, precise control of the biocide solution supplied to each biocide consumer Z1, Z2, BV1 and BV2 can be achieved, even if the biocide solution flows produced in the individual generators differ in some parameters, e.g., having different concentrations of biocide, different volumetric flows or also different temperatures.

Otherwise, the same options as described above for FIG. 1 apply to the embodiment of the apparatus or method according to the invention shown in FIG. 2. In this case, for example, multi-way valves can also be provided at all line branching positions in addition to or as an alternative to the valves V8 to V15, a supply of fresh water can be provided, etc.

Finally, FIG. 3 shows a particularly preferred embodiment in which, in contrast to that in FIG. 2, the valves V1, V2 and V8 to V15 are actually each replaced by multi-way mixers M1 to M6. In the feed lines to the four biocide consumers Z1, Z2, BV1 and BV2, the three-way mixers M3 to M6 are provided instead of valves V8 to V15, and the valves V1 and V2 following the check valves R1 and R2 have been replaced by the three-way mixers M1 and M2, into each of which a feed line for fresh water from the fresh water reservoirs FR1 and FR2 also opens. Of course, as mentioned above in reference to FIG. 1, an additional fresh water feed could also be provided in the multi-way mixers M3 to M6 if they are designed as four-way mixers instead of three-way mixers.

Additionally, FIG. 3 shows storage tanks for aqueous solutions of peroxodisulfate, P1 and P2, and of chlorite, C1 and C2, respectively, which supply both generators G1 and G2 with such biocide precursor solutions via corresponding feed lines, in order to produce the chlorine dioxide solution which is especially preferred according to the invention by the peroxodisulfate chlorite process as biocide solution.

All of the other options described above will also be applicable analogously to the embodiment shown in FIG. 3. Thus, it will be assumed herein, that both fresh water reservoirs FR1 and FR2 as well as the storage tanks for biocide precursor solutions P1, P2, C1 and C2 will each be equipped with an integrated pump, and any multi-way valve could optionally be substituted by a mixing chamber.

A number of other modifications and additions which are necessary or desirable for various applications or processes are also known to those skilled in the art in the treatment of foodstuffs and more particularly of beverages, especially in tunnel-type pasteurizers, and consequently, in combination with the features of the invention disclosed herein, also fall within the scope of the appended claims.

The present invention thus provides a new apparatus and method for treating tightly sealed foodstuff containers by spraying with a biocide-containing treatment liquid, which, compared to the prior art, provide a higher reliability as well as a higher flexibility of the treatment itself.

1. An apparatus for treating tightly sealed foodstuff containers by spraying with a treatment liquid containing at least one biocide in at least one treatment zone equipped with a plurality of spraying means for spraying said containers, the apparatus further comprising transporting means for transporting the containers through the treatment zones, at least one generator for producing a solution of the at least one biocide, at least one heating device for heating the treatment liquid, lines for supplying the biocide solution from the generator to the at least one treatment zone, lines for supplying the treatment liquid to the spraying means, as well as valves required to control the liquid flows, adjustable metering devices and pumps,
characterized in that:
a) wherein said at least one generator comprises at least two generators for producing the biocide solution, and
b) the generators are not connected nor connectable neither directly nor indirectly with one another, but are connected or connectable with any treatment zones and any further biocide consumer devices via corresponding feed lines.

2. The apparatus according to claim 1, characterized in that the feed lines leading from the generators to the treatment zones and any further biocide consumer devices are connected or connectable with one another via mixing valves.

3. The apparatus according to claim 1, characterized in that at the outlet of each generator, a check fitting is provided.

4. The apparatus according to claim 1, characterized in that the treatment liquid is an aqueous solution of the at least one biocide, and the generators are for producing an aqueous solution of the at least one biocide at varying concentrations which are higher than the biocide concentration of the treatment liquid, and each include feed lines for one or more biocide precursor solutions.

5. The apparatus according to claim 4, characterized in that the generators are for producing an aqueous chlorine dioxide solution according to the peroxodisulfate chlorite process and each include feed lines for aqueous solutions of peroxodisulfate and of chlorite from corresponding storage tanks.

6. The apparatus according to claim 1, characterized in that the apparatus it is for pasteurizing foodstuffs enclosed in the containers, and comprising at least one heating device for heating the treatment liquid to more than 60° C.

7. The apparatus according to claim 1, characterized in that the apparatus comprises at least another biocide consumer device, selected from devices for rinsing, washing, filling and sealing of the containers as well as for lubricating the transport means for the containers.

8. The apparatus according to claim 1, characterized in that the treatment zones are parts of a tunnel-type pasteurizer, plate-type pasteurizer or chamber pasteurizer.

9. The apparatus according to claim 1, characterized in that the apparatus further comprises measuring sensors for temperatures and/or concentrations of the liquid flows, which are connected to a controller for controlling the heating devices, metering devices, pumps and/or valves.

10. The apparatus according to claim 1, characterized in that the apparatus further comprises at least one mixing chamber for mixing the biocide solution produced in at least one generator with fresh water and/or for mixing of at least two biocide solutions produced by different generators with one another before entering the corresponding treatment zone or into another biocide consumer device.

11. The apparatus according to claim 1, characterized in that the apparatus further comprises one of more of:
one or more feed lines for fresh water;
means for collecting used treatment liquid;
further heating and cooling devices; and
means for exhausting the atmosphere surrounding the treatment zones.

12. A method for treating tightly sealed foodstuff containers by spraying with a treatment liquid containing at least one biocide using an apparatus according to claim 1, the method comprising the steps:
producing a solution of the at least one biocide in the at least two generators which are neither directly nor indirectly connected to one another;
directing the biocide solution to at least one treatment zone equipped with a plurality of spraying means, each generator being connected or connectable with any treatment zones and any further biocide consumer devices;
mixing the treatment liquid with the biocide solution; and,
spraying the containers with the treatment liquid via the spraying means, while they are being transported through the treatment zones.

13. The method according to claim 12, characterized in that the containers are sprayed with an aqueous solution of at least one biocide, and an aqueous solution of the at least one biocide is produced in the generators at varying concentrations, which are higher than the biocide concentration of the treatment liquid.

14. The method according to claim 12, characterized in that the foodstuff enclosed within the containers are pasteurized with the treatment liquid which is heated to more than 60° C., prior to spraying the containers.

15. The method according to claim 12, characterized in that the biocide concentration of the biocide solutions and the treatment solutions produced in the generators are measured, and that the amounts of each liquid flow supplied to the at least one treatment zone are controlled using the measured values.

16. The method according to claim 12, characterized in that the generators, aqueous biocide solutions are produced at least periodically at varying concentrations.

17. The method according to claim 12, characterized in that treatment liquid is introduced into the individual spraying means of a treatment zone at varying temperatures.

18. The method according to claim 12, characterized in that fresh water is continuously or intermittently supplied and/or the atmosphere surrounding the treatment zones is extracted.

19. The method according to claim 12, wherein, in the generators, an aqueous chloride dioxide solution is produced according to the peroxodisulfite chlorite process from peroxodisulfate and chlorite.

20. The method according to claim 12, wherein biocide solutions from different generators having varying concentrations are mixed with one another prior to entering into a treatment zone and/or into any further biocide consumer device.
